(19) United States
(12) Patent Application Publication
(10) Pub. No.: US 2017/0189338 A1
(43) Pub. Date: Jul. 6, 2017

(54) TABLETING AGENT HAVING A LOW WATER CONTENT, AND METHOD FOR THE PRODUCTION THEREOF

(71) Applicant: Merck Patent GmbH, Darmstadt (DE)

(72) Inventors: James EASSON, Darmstadt (DE); Walter HAMM, Weiterstadt (DE); Guenter MODDELMOG, Reinheim (DE)

(73) Assignee: Merck Patent GmbH, Darmstadt (DE)

(21) Appl. No.: 15/466,187

(22) Filed: Mar. 22, 2017

Related U.S. Application Data
(63) Continuation of application No. 13/383,061, filed on Jan. 9, 2012.

(30) Foreign Application Priority Data
Jul. 10, 2009 (EP) 09009066.3

(51) Int. Cl. A61K 9/20

(52) U.S. Cl. CPC A61K 9/2009; A61K 9/2018; A61K 9/2095

(57) ABSTRACT

The present invention relates to a Tabletting aid with a low water content and to a process for the preparation thereof. The Tabletting aid composition is a directly compressible composition, the use of which results in improved tablet properties.

The present invention relates to a Tabletting aid with a low water content and to a process for the preparation thereof. The Tabletting aid composition is a directly compressible composition, the use of which results in improved tablet properties.

Direct compression (DC) is a simple, rapid, inexpensive and flexible tablet production process which protects the active compound. For various reasons, however, not all components which can be employed for the formulation of tablets are suitable for use in this process.

Thus, for stability reasons, some solid administration forms have to be formulated with basic materials with a particularly low water content. Anhydrous calcium hydrogenphosphate as such is a suitable basic substance here, for example for the preparation of tablet formulations.

Owing to poor flow properties and lack of compressibility, however, pulverulent, anhydrous calcium hydrogenphosphate usually cannot be employed as tablet vehicle in direct tabletting without special additives.

In general, therefore, only anhydrous calcium hydrogenphosphates which have been specifically physically modified are suitable for this process. Owing to their brittle material character, however, the compressibility of these materials is likewise often inadequate in many formulations. In addition, the disintegration times of the pressed tablets produced from these directly compressible, anhydrous calcium hydrogenphosphates are in some cases unsatisfactory, also due to the low solubility of anhydrous calcium hydrogenphosphate in aqueous media. Anhydrous DC calcium hydrogenphosphates also have organoleptic disadvantages owing to the sandy, sharp-edged particle structure and their poor solubility, meaning that their use in orally disintegrating administration forms is restricted. In addition, anhydrous calcium hydrogenphosphates exhibit high ejection forces in the tabletting process, which result in considerable mechanical stressing of the tabletting moulds and machines, together with increased wear of the compression moulds, but also in increased machine loads, which in turn results in undesired down times for repairs or also for the requisite acquisition of replacements. Overall, these disadvantageous properties thus have an adverse effect on the durability and up time of the equipment.

The object of the present invention is thus to provide a process by means of which these problematic active compounds and tabletting aids can also be converted into tablets by direct compression in a process. A further object of the present invention is to prepare from these active compounds and tabletting aids free-flowing, readily compressible compositions which can be pressed into tablets in a simple manner while avoiding the abovementioned disadvantages.

The present object is achieved per se by preparing free-flowing, readily compressible compositions which allow direct compression, even with addition of less readily tablettable formulation components, by skilful combination and/or physical modification of the principal constituents of a tablet formulation. In particular, the properties of the various added components are utilised in such a way that these DC materials can be processed simply, are physiologically and chemically inert and can be converted, even with the lowest possible pressing forces, into tablets having very good tablet hardnesses at the same time as adequately fast disintegration times.

The present invention relates, in particular, to a directly compressible composition for the production of tablets, comprising anhydrous calcium hydrogenphosphate and a flexible Tabletting aid.

This directly compressible composition for the production of tablets consists, in particular, of anhydrous calcium hydrogenphosphate and at least one polyol.

This composition particularly preferably consists of anhydrous calcium hydrogenphosphate and at least one polyol selected from the group mannitol, sorbitol, xylitol and erythritol. This composition particularly preferably comprises anhydrous calcium hydrogenphosphate and the polyols mannitol and sorbitol.

Particularly good properties have been found if this directly compressible composition for the production of tablets is prepared using a combination of 50-85% by weight of anhydrous calcium hydrogenphosphate, 10-40% by weight of mannitol and 5-20% by weight of sorbitol, in particular a combination comprising 50 to 80% by weight of anhydrous calcium hydrogenphosphate, 15 to 25% by weight of mannitol and 7 to 13% by weight of sorbitol.

Particular preference is given to corresponding directly compressible compositions which comprise a combination of 65 to 85% by weight of anhydrous calcium hydrogenphosphate, 17 to 23% by weight of mannitol and 8 to 12% by weight of sorbitol.

Especial preference is given to directly compressible compositions for the production of tablets which consist of a combination of 60 to 80% by weight of anhydrous calcium hydrogenphosphate, 15 to 25% by weight of mannitol and 5 to 15% by weight of sorbitol. Corresponding compositions in which anhydrous calcium hydrogenphosphate is present in an amount of 65 to 75% by weight, mannitol is present in an amount of 17 to 23% by weight and sorbitol is present in an amount of 8 to 12% by weight and these components have been co-spray-granulated with one another also have advantageous properties.

Directly compressible, co-spray-granulated compositions according to the invention, as described here, can be metered very well both for tabletting and for the production of capsules since they have a favourable flow angle in the range from 29 to 33.4°. Since these compositions have bulk densities in the range from 0.56 to 0.77 g/ml and tamped densities in the range from 0.73 to 0.92 g/ml, they can be converted particularly well into tablets having comparatively high tablet hardnesses. In this connection, the particle-size distribution in the directly compressible compositions is particularly advantageous; more precisely, compositions according to the invention have a particle-size distribution with max. 3% by weight of undersized particles having a particle size of <32 μm, max. 5% by weight of oversized particles having a particle size of >500 μm, and 50 to 90% by weight of a particle fraction having particle sizes in the range from 100 to 315 μm. The present invention thus also relates to a directly compressible composition which has a calcium content of 14 to 21% by weight, based on the total amount, and a drying loss of less than 2% by weight, in particular less than 1% by weight. The directly compressible compositions found here can advantageously be pressed by compression with a pressing force of 20 kN to give tablets having hardnesses of >270 N which require an ejection force of <215 N, have a friability of <0.16% and at the same time exhibit a disintegration time of <580 seconds. In particular, they can be shaped by compression with a pressing force of 20 kN to give pressed tablets having hardnesses of >300 N, together with an ejection force of <100 N, a friability of <0.16% and a disintegration time of <580 seconds. Increasing the pressing force to 30 kN gives pressed tablets having hardnesses of >350 N, together with an ejection force of <115 N, a friability of at most 0.14% and a disintegration time of <550 seconds. The present invention thus also relates to a composition or formulation which comprises this compressible composition and is in solid form or in the form of a compressate. A composition or formulation of this type may comprise one or more homogeneously distributed, water-insoluble and/or water-soluble additives. These additives are preferably selected from the group pharmaceutical active compounds, plant extracts, sweeteners, dyes, citric acid, vitamins and trace elements. Furthermore, such a composition or formulation according to the invention may comprise one or more pharmaceutical active compounds from the group of the analgesics, but, in particular, also one or more sweeteners selected from the group acesulfame K, Aspartame®, saccharin, cyclamate, sucralose and neohesperidin DC.

The present invention also relates to a process for the preparation of directly compressible compositions for the production of tablets in which a solution or suspension comprising 50 to 85% by weight of anhydrous calcium hydrogenphosphate, 10 to 40% by weight of mannitol and 5 to 20% by weight of sorbitol in water, preferably 60 to 80% by weight of anhydrous calcium hydrogenphosphate, 15 to 25% by weight of mannitol and 7 to 13% by weight of sorbitol in water, where 4 parts of solid are dissolved or suspended in 4 parts of water, is subjected to a co-spray-granulation process, either batchwise or continuously in a fluidised-bed granulator. Experiments have shown that the combination of brittle, anhydrous calcium hydrogenphosphate with a rather flexible material, such as, for example, a polyol, results in significantly improved tablet quality, which is on the one hand evident from considerably improved compressibility, but on the other hand tablets having a fast tablet disintegration time are simultaneously obtained. In particular, it has been found that a correspondingly improved product can be obtained from a combination consisting of a co-spray-granulated composition comprising about 50-85% by weight of anhydrous, pulverulent calcium hydrogenphosphate, about 10-40% by weight of mannitol and about 5-20% by weight of sorbitol. In particular, a co-spray-granulation process gives a product for the direct-tabletting process which is optimum with respect to flow behaviour, compressibility, disintegration properties and other pharmaceutical formulation characteristics. The material according to the invention exhibits significantly better processing properties than would be possible, for example, by simple physical mixtures, even using directly tablettable individual components. It has furthermore been found that the pharmaceutical formulation properties of these co-sprayed products are only improved by the addition of a certain amount of sorbitol.

In the production of tablets, in particular in the case of active compounds which are sensitive to moisture, it must be ensured that as far as possible no water is introduced by the tabletting aids employed in a pharmaceutical formulation.

In addition, the ratio of the three constituents mentioned above must be kept within an optimised range in order to obtain the improved pressing force/hardness or hardness/disintegration time profiles. In particular, it has been found that the improved properties are obtained if the weight ratio is in a range between about 50:40:10 and 70:20:10, based on the ratio of anhydrous calcium hydrogenphosphate employed to mannitol to sorbitol. In this range, the corresponding compositions give particularly improved pressing force/hardness or hardness/disintegration time profiles. This composition apparently has a balanced ratio between the flexibility of the polyols and the brittleness of the anhydrous calcium hydrogenphosphate, which produces the very good pressing properties.

In order to improve the compressibility of anhydrous calcium hydrogenphosphate at the same time as a fast tablet disintegration time and the lowest possible ejection forces during processing, it has been found that the combination of brittle anhydrous calcium hydrogenphosphate with a comparatively flexible material, such as, for example, a polyol, significantly improves the resultant tablet quality.

In particular, it has been found that co-spray-granulation of a combination of about 70% by weight of pulverulent, anhydrous calcium hydrogenphosphate with about 20% by weight of mannitol and about 10% by weight of sorbitol gives a product for the direct-tabletting process which is optimum with respect to flow behaviour, compressibility, disintegration properties and ejection force. The two polyols contain no water of crystallisation and thus introduce virtually no additional water components into the formulation. Furthermore, it is possible, under the co-spray-granulation conditions described, to obtain material with a low water content having a drying loss of <1% by weight. The material according to the invention exhibits significantly better processing properties than could be obtained, for example, by simple physical mixtures of the corresponding individual components with a low water content. Compared with an anhydrous calcium hydrogenphosphate which is relatively suitable for direct tabletting, which is commercially available under the trade name “Fujicalin”, the compositions prepared in accordance with the invention have improved properties. Thus, the tabletting properties with respect to pressing force/hardness, hardness/disintegration time, and very particularly with respect to the requisite ejection forces, are improved at higher pressing forces.

Surprisingly, it has been found that the pharmaceutical formulation properties of the resultant co-sprayed product are improved, in particular with the addition of sorbitol, in particular if the three components mentioned above are used in the optimum ratio to one another. A balanced ratio between flexibility (of the polyols) and brittleness (of the anhydrous calcium hydrogenphosphate), which produces the very good pressing properties, apparently exists in this composition.

The aim of the preparation of the compositions according to the invention having improved tabletting properties must thus be to prepare a product having a very homogeneous distribution of the anhydrous calcium hydrogenphosphate, which is virtually insoluble in water at a neutral pH, in a matrix of the two water-soluble polyols mannitol and sorbitol. This homogeneous distribution is achieved, as the experiments have shown, by a co-spray-granulation process of all components from aqueous solution or suspension in a fluidised bed.

In order to obtain this highly homogeneously distributed, anhydrous calcium hydrogenphosphate in the polyol matrix by co-spray-granulation, starting granules are firstly produced (pre-spraying), for example in a batch process, and then serve in the form of a small amount for initial introduction in the fluidised bed for one or more further co-spray-granulation processes (main sprayings). In this way, the proportion of inhomogeneously distributed anhydrous calcium hydrogenphosphate in the polyol matrix can continue to be reduced down to a negligible proportion. An optimum homogeneous distribution of the anhydrous calcium hydrogenphosphate in the polyol matrix is ideally obtained if a product which has a homogeneous distribution is initially introduced in the fluidised-bed granulator right at the beginning of the co-spray-granulation. In this case, the pre-sprayings are superfluous.

In continuous operation of the fluidised-bed granulator, the constant removal of co-spray-granulated product and partial recycling of formed product means that the process is carried out in such a way that the process is at equilibrium after a certain time and the homogeneously distributed anhydrous calcium hydrogenphosphate desired is obtained in the polyol matrix. If a product which has a homogeneous distribution is initially introduced in the fluidised-bed granulator right at the beginning, the time from start-up of the continuous process to equilibrium is naturally shortened.

In the material obtained in this way, the formulation pharmacist is provided with a product which is optimised with respect to the direct-tabletting properties and with the aid of which active compounds which are poorly tablettable per se and also those which are sensitive to moisture are also able to undergo this simple tabletting process. In addition, its high calcium and phosphate content means that the product can be employed for the formulation of calcium- and phosphorus-enriched pressed tablets, for example in chewable tablets for food supplementation. This use is also particularly appropriate since the material gives rise to very good sensory properties due to the extremely fine distribution of the naturally sandy-tasting anhydrous calcium hydrogenphosphate in a matrix of the sweet- and cool-tasting polyols. A composition which, besides the pleasant sensory properties (mouth feel), also has significantly improved direct-compression properties is thus provided. Furthermore, the material has a very low drying loss.

In this connection, the co-spray-granulated compositions according to the invention, prepared from anhydrous calcium hydrogenphosphate, mannitol and sorbitol, exhibit a number of unexpected advantages:
1. Very good direct-compressibility properties:
   Whereas anhydrous calcium hydrogenphosphate and mannitol can usually be pressed to give tablets with some difficulty, the three-component combination of anhydrous calcium hydrogenphosphate, mannitol and sorbitol after co-spray-granulation has very good properties during direct compression, in particular if the components in certain mixing ratios are subjected to co-spray-granulation with one another in advance. The products obtained in this way can subsequently be converted into tablets having improved properties.
   In the experiments, the best pressing force/hardness ratios were achieved at a ratio of the three components to one another of 70:20:10, 60:30:10 and 50:40:10. In the case of combinations having a ratio of the individual components to one another in this range, ratios, which are optimum for compression, of the brittle properties of the anhydrous calcium hydrogenphosphate and the flexible properties of a mannitol/sorbitol combination apparently exist. In particular, a combination in the ratio of about 70:20:10 exhibits the hardest compressates at relatively high pressing forces without a tendency towards capping and with low friability.
   Co-spray-granulated compositions having a relatively high content of anhydrous calcium hydrogenphosphate or also compositions in which anhydrous calcium hydrogenphosphate has been co-spray-granulated only with mannitol or only with sorbitol exhibit worse compressibilities.
   Compared with commercially available directly compressible (DC) anhydrous calcium hydrogenphosphate grades, the direct-compression properties of the co-spray-granulated three-component combinations according to the invention are improved. This likewise applies in comparison to a physical mixture of anhydrous commercially available DC calcium hydrogenphosphate with the DC mannitol grade which is very readily directly compressible per se (Parteck® M 200) and DC sorbitol (Parteck® SI 150) in the weight ratio 70:20:10.
   Only the anhydrous calcium hydrogenphosphate which is commercially available under the name Fujicalin® exhibits a similar pressing force/hardness profile, but the co-spray-granulated compositions according to the invention having a ratio of the individual components to one another in the range from 60:30:10 and 70:20:10 also have improved tablet hardnesses compared with Fujicalin®, and at relatively high pressing forces.
2. Fast disintegration times of the pressed tablets, even in high hardness ranges
   Short disintegration times are still achieved at the same time as high tablet hardnesses, even without the addition of disintegration-promoting components (disintegrants), through the co-spray-granulation as a three-component combination of 50 to 85% by weight of anhydrous calcium hydrogenphosphate with preferably at least two polyols, particularly preferably mannitol and sorbitol. Addition of a larger amount of anhydrous calcium hydrogenphosphate results in a retardation of the disintegration time.
   In the experiments, short disintegration times were exhibited, in particular, by the co-spray-granulated three-component combinations of anhydrous calcium hydrogenphosphate, mannitol and sorbitol in the weight ratio 70:20:10 over a significantly broader tablet hardness range, to be precise significantly shorter than commercially available anhydrous DC calcium hydrogenphosphate or compared with the physical mixture of the individual components of DC materials. Only the Fujicalin® mentioned above exhibits shorter disintegration times, but together with a less favourable pressing force/hardness profile. In particular, the high tablet hardnesses from Examples E (70:20:10) and D (60:30:10) cannot be achieved with Fujicalin®.
3. Low dry residue
   Compositions according to the invention advantageously exhibit low drying losses of <1% by weight (at 105° C. for 3 h). This indicates that the co-spray-granulation process also does not bind any free water.
   The compositions according to the invention are thus suitable for the formulation of active compounds and tabletting aids which are also sensitive to moisture.
4. Mechanical stability of the resultant tablets
   The mechanical stability of pharmaceutical formulations in the form of granules or tablets is assessed, inter alia, with reference to their friability. Friability is a measure in percent by weight of the mechanical abrasion of the tablets under mechanical load. Tablets are subjected to physical loads from production to final consumption. They must therefore be developed in such a way that they survive the impacts arising with as little damage as possible.
   In order to establish how the tablets to be tested survive these loads and in order to be able to draw conclusions on further processing (for example coating, sugar coating, packaging), friability or abrasion tests are therefore carried out. In these tests, the tablets are tested in accordance with the principle of a recurring movement in Roche friabilator or abrasion drums. The test conditions, such as number of samples, number of rotations and test speed, are defined in the pharmacopoeias. Abrasion is defined as the mass that the tablets lose due to the mechanical load.
   In order to determine the friability, various instruments in different designs are commercially available. The ERWEKA TDR 100 tester is a semi-automatic combination system comprising an ERWEKA abrasion/friability tester and a Sartorius analytical balance (Erweka Apparatebau, Heusenstamm).
   Other instruments are:
   Model TAP No. 43651 friability tester, Erweka Apparatebau, Heusenstamm
   Abrasion tester from Arzneimittelwerk Dresden, Dresden,
   Friabilator model PTF 1, Pharmatest,
   Roche friabilator, J. Engelsmann AG, Ludwigshafen/Rhein
   The friability of the tablets to be tested is tested in these instruments by methods as described in Ph. Eur. Supplement 2001 or Ph. Eur. 6th Edition, main part 2008, under “2.9.7 Friability of uncoated tablets”.
   For the assessment, a fixed number of dust-free tablets can be agitated in a drum with a chicane for a certain time and at a fixed speed. The mass loss of the dust-freed tablets is subsequently determined in percent.
   In the experiments of the present invention, the friability of the tablets produced by pressing was determined by investigating the abrasion of tablets in a Roche friabilator as described in Ph. Eur. 6th Edition, main part 2008, under 2.9.7. In each case, 100 rotations were carried out with the instrument, with the rotational speed being 25+/−1 min⁻¹.
   The friabilities of the compositions according to the invention, consisting of the three-component combination in a weight ratio in the range between 50:40:10 and 70:20:10, in particular the co-spray-granulated compositions investigated in the ratio 50:40:10, 60:30:10 and 70:20:10, are significantly reduced in the case of all pressing forces tested compared with commercially available anhydrous DC calcium hydrogenphosphate grades. No “capping” is observed for these compositions during pressing. These compositions thus enable very safe handling of the tablets produced in the further processing, for example in packaging machines or coating equipment, or on removal from blister packs by the patient. Only pressed products based on Fujicalin® exhibit very good friabilities, but they do not achieve the higher hardnesses of Examples D and E at the higher pressing forces.
5. Low ejection forces:
   The ejection forces for compositions C, D, E investigated, in particular for composition E having a weight ratio of the individual components to one another of 70:20:10, are reduced at all pressing forces tested with high tablet hardnesses compared with those of the commercially available directly compressible anhydrous calcium hydrogenphosphates. This thus results in optimum protection of the compression moulds (punch moulds) and the tabletting machines on use of the compositions according to the invention.
   In particular, Fujicalin® exhibits significantly increased ejection forces at all pressing forces, which is an indicator of increased mechanical stressing of the tabletting moulds.

On use of compositions according to the invention during tabletting, these stuck neither to the punch nor to the dies of the tabletting machine nor between the punch and the dies. The compositions according to the invention also have no tendency to accumulate on the punches and dies and thus cause friction between the punch and the die. They can therefore be ejected from the tabletting dies with reduced pressure.

The compositions according to the invention can be processed industrially in a continuous and stable manner for a long time on corresponding tabletting machines without so-called rough running of the machines occurring.
6. High calcium hydrogenphosphate content:
   Simply the preparation of the precursors for the production tablets by co-spray-granulation of anhydrous calcium hydrogenphosphate, mannitol and sorbitol in the weight ratios according to the invention gives compositions having a high calcium hydrogenphosphate content, in particular by co-spray-granulation in the weight ratio 70:20:10. These compositions can therefore be used for calcium and phosphorus enrichment in foods, food supplements or in pharmaceutical preparations, in particular in the form of powder sachets, pressed tablets or in capsules. Furthermore, the flow angle of the material prepared is optimal for further processing, in the range from 29 to 33.4°. The material is thus, for example, eminently suitable for individual metering into the dies of the tabletting machines during tabletting or for sachet filling by machine.
7. Good taste properties:
   The extremely fine distribution of the fine calcium hydrogenphosphate in the cooling- and sweet-tasting polyol matrix masks the unpleasant sandy feel in the mouth of the calcium hydrogenphosphate, which is virtually insoluble in water at a neutral pH, so that use of the compositions according to the invention improves customer or patient compliance.
8. Remark on Fujicalin®:
   The experiments have shown that commercially available Fujicalin® comes closest to the co-spray-granulated compositions according to the invention in its pharmaceutical formulation properties. However, the compositions according to the invention have significant improvements compared with Fujicalin®.
   Fujicalin® has
   a) as already stated above, impaired compressibility at relatively high pressing forces (20 and 30 kN), in particular compared with compositions having the weight ratios 70:20:10 (E) and 60:30:10 (D),
   b) the highest ejection forces of all samples tested at all 4 pressing forces tested, even taking into account the achievable tablet hardnesses.

In order to carry out the co-spray-granulation, aqueous solutions or suspensions of the various components are prepared in advance. 50% [50% (w/w)] solutions or suspensions are preferably used, where the percent data relate to the respective percentages by weight. The dry substances are dissolved or suspended in demineralised water. In order to carry out the co-spray-granulation, the amount ratios of the anhydrous calcium hydrogenphosphate to the polyols mannitol and sorbitol are adjusted in solution in such a way that the desired ratios by weight arise in the ratio to one another according to the invention in the co-sprayed substance. For the preparation of the spray solution, the amount of demineralised water calculated in advance is initially introduced in a batch vessel. The polyols sorbitol and mannitol are stirred into the water at 20-25° C. until completely dissolved. The calcium hydrogenphosphate is introduced into this clear solution, likewise with stirring, and the white suspension is stirred until any agglomerates formed have disintegrated. This solution/suspension is sprayed with constant stirring in the co-spray-granulation.

In order to obtain a calcium hydrogenphosphate with the most homogeneous distribution possible in the polyol matrix, starting granules may firstly be produced in a batch process (pre-spraying), of which in each case a small amount can then be employed for initial introduction in the fluidised bed for one or more co-spray-granulation processes (main sprayings). In this way, the proportion of inhomogeneously distributed calcium hydrogenphosphate in the polyol matrix can continue to be reduced to a negligible proportion.

It is of course significantly simpler if the starting granules employed comprise co-sprayed material of the desired composition which has been removed from prior sprayings and can be initially introduced in the fluidised bed. The spraying is then carried out as described for the main spraying in the following examples.

The continuous preparation is carried out in a similar manner as described in the specifications EP 1 453 781 A1, EP 1 319 644 A1 and WO 00/76650 A1, for example for the preparation of alpha- or beta-mannitol. In particular, the continuous preparation of the compositions according to the invention is carried out in a fluidised-bed granulator with powder recycling and continuous removal of product, where the average particle size of the resultant product is controlled by the air stream in the fluidised bed.

This co-spray-granulation produces a very homogeneous distribution of the anhydrous calcium hydrogenphosphate, which is virtually insoluble in water at a neutral pH, in a matrix comprising the two water-soluble polyols mannitol and sorbitol. This homogeneous distribution is produced by a co-spray-granulation process of all components from aqueous solution or suspension in a fluidised bed. Besides a product having pleasant sensory properties (mouth feel), a product having very good direct-compression properties is also obtained in this way.

The principle of the co-spray-granulation process and the design of the equipment can be obtained from the patent specifications EP 1 453 781 (beta-mannitol), EP 1 319 644 (alpha-mannitol) and WO 00/76650.

The desired particle sizes can be produced by variation of the process parameters spray pressure, spray amount, recycled amount of powder, hot-air stream and hot-air temperature. If necessary, a particle-size restriction can also be carried out by sieve classification at the discharge. Coarse particles can be recycled into the spray system after comminution by a grinding fan.

In principle, the compositions according to the invention are prepared in a similar manner as described in the specifications EP 1 453 781A1, EP 1 319 644 A1 for the preparation of alpha- and beta-mannitol or in WO 00/76650 A1. More precisely, the co-spray-granulation is carried out in a fluidised-bed granulator with powder recycling, in which the spraying of the solutions or suspensions is carried out by means of two-component nozzles, via which powder recycled simultaneously is transported into the spray zone.

For this purpose, the spray pressure of the two-component nozzles should be set in the range 2-4 bar, preferably in the range 2.5-3.5 bar. The amount of hot gas fed to the two-component nozzle should be regulated in such a way that up to about 1.5-3 m³/(h kg of suspension) is conveyed at a temperature of about 80-110° C.

The powder recycling should be set in such a way that solids recycling takes place in the range 0.2-2.0 kg of solid/(h kg of suspension), preferably in the range 0.5-1.5 kg of solid/(h kg of solution). The process is particularly favourable if the solids recycling is in the range 0.5-1.0 kg/(h kg of solution).

In order to carry out the process, pre-warmed air must be fed into the equipment. Good results are achieved if the air fed to the equipment is pre-warmed to a temperature in the range 45-120° C. It is favourable for the process according to the invention for the feed air to have a temperature in the range 65-110° C. It is particularly advantageous for the formation of co-spray-granulated calcium hydrogenphosphate/mannitol/sorbitol having good tabletting properties if the temperature of the air fed in is in the range 70-100° C. The amount of feed air supplied should be regulated in accordance with the invention in such a way that 1000-2000 m³/m² per hour, in particular 1200-1700 m³/m² per hour, are fed into the equipment.

In combination with the other parameters set, favourable process conditions exist if the air stream in the equipment is guided in such a way that the exhaust-air temperature becomes established in the range 30-50° C. and the temperature of the product formed becomes established at a temperature in the same range up to 50° C.

It has furthermore proven favourable to regulate the process conditions in such a way that the amount of powder located in the fluidised bed becomes established at an amount of bed of 50-150 kg/m² of bed. It is particularly favourable for the amount of bed to be in the range 80-120 kg/m² of bed.

Targeted powder recycling both by powder removal from the fluidised bed and also by recycling of a very fine powder fraction formed during formulation, i.e. homogenisation of the particle size by sieving during packaging of the product produced, enables the process to be controlled with respect to the desired particle-size distribution.

It is also possible to comminute powders having relatively large particle cross sections in the grinding fan of the spray-granulation unit before the recycling so long as a fluidised-bed granulator is used, as described in EP 1 453 781 A1 or EP 1 319 644 A1.

The special preparation procedure in a co-spray-granulation process gives directly compressible compositions having a bulk density in the range from 0.56 to 0.77 g/ml with a tapped density in the range from 0.73 to 0.92 g/ml. These properties are combined with a particle-size distribution of max. 3% by weight of undersized particles having a particle size of <32 μm, max. 5% by weight of oversized particles having a particle size of >500 μm, and 50 to 90% by weight of a particle fraction having particle sizes in the range from 100 to 315 μm. Depending on the proportion by weight of co-sprayed polyol during the co-spray-granulation, the composition has a calcium content in the range from 14 to 21% by weight, based on the total amount, and a drying loss of less than 2% by weight, in particular less than 1% by weight.

Investigations of the tabletting properties of the directly compressible compositions according to the invention have shown that the composition according to the invention can be compressed with a pressing force of 20 kN to give tablets having hardnesses of >270 N, together with an ejection force of <215 N, a friability of <0.16%, a disintegration time of <580 seconds. If, by contrast, the composition according to the invention is compressed with a pressing force of 30 kN, the pressed tablets have hardnesses of >350 N, together with an ejection force of <115 N, a friability of at most 0.14% and a disintegration time of <550 seconds. Due to a flow angle in the range from 29 to 33.4°, the compositions according to the invention can be metered particularly well in pharmaceutical formulations.

In accordance with the invention, the directly compressible composition in accordance with the present invention can be introduced into a composition or formulation which is in solid form or in the form of a compressate. As pharmaceutically usable composition or formulation, this may in turn comprise one or more homogeneously distributed, water-insoluble and/or water-soluble additives. The homogeneous distribution can be produced either by prior intensive mixing with the directly compressible composition before the tabletting or packaging is carried out. However, the homogeneous distribution can also be achieved by joint co-spray-granulation under suitable conditions. The water-soluble or water-insoluble additives are selected, in particular, from the group pharmaceutical active compounds, plant extracts, sweeteners, dyes, citric acid, vitamins and trace elements. These additives are selected so that they are stable and capable of storage in the combination of the individual components of the composition. In particular, such a composition or formulation according to the invention may comprise one or more pharmaceutical active compounds from the group of the analgesics, but furthermore one or more sweeteners, selected from the group acesulfame K, Aspartame®, saccharin, cyclamate, sucralose and neohesperidin DC, may also be added in order to improve the taste.

For better understanding and in order to illustrate the invention, examples are given below which are within the scope of protection of the present invention. These examples also serve to illustrate possible variants. Owing to the general validity of the inventive principle described, however, these examples are not suitable for reducing the scope of protection of the present application to these alone.

Should anything be unclear, the references and patent specifications cited in the description, which are hereby incorporated into the description of the present invention as part of the disclosure, should also be used for better understanding.

The temperatures given in the examples and description and in the claims are always in ° C. Unless stated otherwise, content data are quoted as % by weight or weight ratios.

It furthermore goes without saying to the person skilled in the art that, both in the example given and also in the remainder of the description, the component amounts present in the compositions always only add up to 100% by weight or mol-%, based on the composition as a whole, and cannot exceed this, even if higher values could arise from the percent ranges indicated. Unless indicated otherwise, % data are taken to be % by weight, with the exception of ratios, which are shown in volume data.

### BRIEF DESCRIPTION OF THE DRAWINGS

FIG. 1 shows a comparison of the pressing force/hardness profiles for the co-sprayed compositions investigated comprising anhydrous calcium hydrogenphosphate, mannitol and sorbitol.

FIG. 2 shows disintegration time vs. hardness of tablets with compositions according to the invention compared with the pure substances.

FIG. 3 shows a comparison of the compositions according to the invention with the tabletting properties of commercially available directly tablettable calcium hydrogenphosphate anhydride grades.

FIG. 4 shows disintegration time vs. hardness of tablets with other compositions according to the invention compared with the pure substances.

FIG. 5 shows a plot of the hardness of the tablets produced vs. needed ejection force.

### EXAMPLES

In order to carry out the co-spray-granulation according to the invention, the following instruments and processes are employed for characterisation of the substance properties:
1. Bulk density: in accordance with DIN EN ISO 60: 1999 (German version)
   data in the tables as “g/ml”
2. Tapped density: in accordance with DIN EN ISO 787-11: 1995 (German version)
   data in the tables as “g/ml”
3. Angle of repose: in accordance with DIN ISO 4324:1983 (German version)
   data in the tables as “degrees” (°)
4. Hausner factor: calculation in accordance with Ph. Eur. 6th Edition, main part 2008, section 2.9.36 “Compressibility index and Hausner factor”
5. Compressibility index: calculation in accordance with Ph. Eur. 6th Edition, main part 2008, 2.9.36, “Compressibility index and Hausner factor”
   data in the tables as “%”
6. Tabletting testing: 492.5 g of the material whose tabletting properties are to be tested are mixed with 7.5 g of Parteck LUB MST (vegetable magnesium stearate) EMPROVE exp Ph. Eur., BP, JP, NF, FCC, Art. No. 1.00663 (Merck KGaA, Germany); the magnesium stearate is passed through a 250 μm sieve in advance and mixed for 5 minutes in a sealed stainless-steel container (capacity: about 2 l, height: about 19.5 cm, diameter: about 12 cm; external dimensions) in a laboratory tumble mixer (Turbula, Willy A. Bachofen, Switzerland). Pressing to give 500 mg tablets (11 mm punch, round, flat, with bevel) is carried out in an instrumented Korsch EK 0-DMS cam tabletting machine (Korsch, Germany) with Catman 5.0 evaluation system, Hottinger Baldwin Messtechnik—HBM (Germany).
   Depending on the pressing force (nominal settings: 5+/−1, 10+/−1, 20+/−2 and 30+/−2 kN; the effectively measured actual values are shown in the examples), at least 100 tablets are produced for evaluation of the pressing data and the pharmaceutical formulation characteristics.
7. Determination of the tablet hardness, diameter and height: Erweka TBH 30 MD; Erweka (Germany); average data from 20 tablet measurements per pressing force
8. Tablet abrasion: friability tester, Erweka (Germany); instrument parameters and performance of the measurements in accordance with Ph. Eur. 6th Edition, main part 2008, 2.9.7. “Friability of uncoated tablets”
9. Tablet weight: average value from the weighing of 20 tablets; balance: Mettler AT 201, Mettler (Germany)
10. Tablet disintegration: disi4 automatic disintegration tester from Biomation (Germany); medium: desalinated water at 37° C.; instrument parameters and performance in accordance with Ph. Eur. 6th Edition, main part 2008, 2.9.1 “Disintegration time of tablets and capsules” (with disc)
11. Determination of the particle sizes as dry sieving via a Retsch AS 200 control ‘g’ sieve tower, Retsch (Germany); amount of substance: 40 g+/−2 g; sieving time: 30 minutes; amplitude: 1 mm; interval: 5 seconds; diameter of the sieve inserts used: 200 mm; sieve sizes: 1000, 710, 500, 315, 200, 100, 50 and 32 μm; amount distribution per sieve fraction indicated in the tables as “% by weight of the sample weight”
12. Calcium content determination: complexometric titration using Na EDTA solution and potentiometric indication or colour indication. The principle of the procedure is described in the technical literature, such as, for example, in G. Jander, K. F. Jahr, H. Knoll “Maβanalyse—Theorie und Praxis der klassischen und der elektrochemischen Titrierverfahren” [Volumetric Analysis—Theory and Practice of Classical and Electrochemical Titration Methods], publisher Walter de Gruyter, 1973 ISBN 3 11 005934 7, or in the application documents from the titration and indicator electrode manufacturers, for example from Mettler-Toledo GmbH, Germany, or Metrohm, Switzerland.
   Before the titration, the samples (sample weight about 0.2 g, weighed accurately) are slurried with a little demineralised water and dissolved using 5 ml of 25% hydrochloric acid. 20.00 ml of 0.1 mol/I Titriplex(III) solution (Art. No. 1.08431; MERCK KGaA, Germany) are metered in, the mixture is made up to 70 ml with demineralised water, a buffer tablet (Art. No. 1.08430, MERCK KGaA, Germany) is added, and, after the buffer tablet has dissolved, the pH is adjusted to 10-11 with stirring using about 10 ml of ammonium buffer solution pH 10-11 (Art. No. 1.09478, MERCK KGaA, Germany). The mixture is subsequently back-titrated potentiometrically with a zinc sulfate solution (0.1 mol/I). The calcium content can be calculated stoichiometrically from the consumed amount of 0.1 mol/I Titriplex(III) solution.
13. Drying loss: about 1.000 g of substance (weighed accurately) is dried for 3 h at 105° C. in a drying cabinet. The arithmetic mean of two independent measurements is quoted as the drying loss.

### Raw Materials for the Preparation of the Examples According to the Invention

Anhydrous calcium hydrogenphosphate, very finely powdered, suitable for use as excipient EMPROVE® exp Ph. Eur., BP, USP, FCC, E 341 (Art. No. 1.02144, Merck KGaA, Germany)

Particle size: 99%<63 μm, measured by laser diffraction with wet dispersal

### Instrument/Method:

Malvem Mastersizer 2000, Hydro 2000 S wet module

### Sample Preparation:

about 500 mg of substance are dispersed in about 50 ml of aqueous, saturated and filtered calcium hydrogenphosphate solution for 1 min in an ultrasound bath

### Evaluation Model:

Universal; medium saturated calcium hydrogenphosphate solution

Refractive index medium 1.35 (MIE parameters); Fraunhofer; stirring speed 2000 rpm

### Ultrasound:

100%, obscuration 10-15%, measurement duration 7500 ms; performance in accordance with the technical manual and instrument manufacturer's specifications

D(−)-mannitol suitable for use as excipient EMPROVE® exp Ph. Eur., BP, USP, JP, FCC, E 321 (Art. No. 1.05980, Merck KGaA, Germany)

Parteck® SI 400 (sorbitol) suitable for use as excipient EMPROVE® exp Ph. Eur., BP, NF, E 420 (Art. No. 1.03140, Merck KGaA, Germany)

Demineralised water

### Comparative Substances

Parteck® M200 (mannitol) suitable for use as excipient EMPROVE® exp Ph. Eur., BP, JP, USP, E 421 (Art. No. 1.00419, Merck KGaA, Germany)

Parteck® SI 150 (sorbitol) suitable for use as excipient EMPROVE® exp Ph. Eur., BP, JP, NF, E 420 (Art. No. 1.03583, Merck KGaA, Germany)

Anhydrous Emcompress® dibasic calcium phosphate, anhydrous, USP, calcium hydrogenphosphate, anhydrous, Ph. Eur. (JRS PHARMA GmbH&Co.KG, Germany), Batch No.: 1046

DI-CAFOS A dicalcium phosphate anhydrous coarse white powder, USP, FCC, Ph. Eur., JP, E 341 (Product No.: C 92-12, Chemische Fabrik Budenheim KG, Germany)

Material No. 00000589, Batch No.: A95505A

A-TAB® dicalcium phosphate, anhydrous, granular USP, EP, FCC, E 341 (Innophos Inc., USA; purchased via Univar GmbH, Essen, Germany) batch 2700

DI-CAFOS AN dicalcium phosphate anhydrous coarse powder, USP, FCC, Ph. Eur., E 341 (product No.: C 92-22, Chemische Fabrik Budenheim KG, Germany), Material No. 00005231, Batch No.: A67665A

FUJICALIN™SG dibasic calcium phosphate anhydrous DCPA, USP/NF, EP, JP (FUJI CHEMICAL INDUSTRY CO., LTD, Japan; purchased via SEPPIC GmbH, Cologne, Germany), batch No. CP 612006

### General Performance of Co-Spray-Granulation

A solution or suspension of 4 parts of water and 4 parts of solid, where the solid consists of 7 parts of pulverulent, anhydrous calcium hydrogenphosphate, 2 parts of mannitol and 1 part of sorbitol, or where the solid has the ratio of the pulverulent starting materials anhydrous calcium hydrogenphosphate, mannitol and sorbitol in the desired ratio of the composition to be prepared, is subjected to a co-spray-granulation process (batchwise or continuous) in a fluidised-bed granulator. In order to prevent adhesive effects, in particular also on use of a continuous process, partial solids recycling can be employed. Product having a defined particle-size distribution or bulk and tapped density can be obtained by suitable operation of the equipment or by a subsequent sieving process.

### Co-Spray-Granulation in Batch Operation (Laboratory)

### Note:

The raw materials employed, the amounts thereof, and the quantitative compositions of the co-spray-granulated end products are shown in the tables.

### Preparation of Spray Solutions or Spray Suspensions:

All spray solutions and suspensions were prepared with 50% (w/w) of dry substance in demineralised water, as indicated in the general example. The ratios of the anhydrous calcium hydrogenphosphate to the polyols arise from the desired compositions of the desired end products, as indicated in Table 1.

The polyols are stirred into the water initially introduced in a batch vessel at 20-25° C. until completely dissolved. The anhydrous calcium hydrogenphosphate is introduced into this clear solution, likewise with stirring, and the white suspension is stirred until any agglomerates formed have disintegrated. This solution/suspension is sprayed with constant stirring.

### Preparation of the Starting Material for the Co-Sray-Granulation:

In order to start up the equipment, it is necessary to pass a primary amount of bed through the co-spray-granulation process. This starting bed can be produced in two ways:
1. The equipment is filled at the beginning of the spraying process with material retained from material previously co-sprayed in accordance with the invention
   or
2. The equipment is filled with a physical mixture of the desired components in the qualitative and quantitative composition to be prepared, i.e. with pulverulent anhydrous calcium hydrogenphosphate, mannitol and sorbitol. The co-spray-granulation process is carried out as described, but without removing material at the outlet. Instead, all the material is recycled into the process via a grinding fan until a stable process and a product composition according to the invention has been achieved. The establishment of the particle-size distribution is then begun with product removal, as described for a continuous process.

In order to illustrate the corresponding procedure, the example which results in the preparation of the co-sprayed composition called product E below is described. In order to obtain a starting material which can be employed for the co-spray-granulation, a suitable precursor is prepared by pre-spraying and can then be initially introduced in the fluidised-bed granulator for the actual co-spray-granulation, the main spraying:

### 1. Pre-Spraying:

0.20 kg of mannitol and 0.25 kg of sorbitol are added to 2.20 kg of demineralised water at 20-25° C. with stirring. When a clear solution has been obtained, 1.75 kg of anhydrous calcium hydrogenphosphate are added and suspended with stirring.

0.3 kg of mannitol powder is initially introduced in a GPCG 5 fluidised-bed granulator (Glatt, Germany) or as described in WO 00/76650 A1, and fluidised. The spray suspension is sprayed onto this fluidised bed. In order to carry out the co-spray-granulation, the instrument parameters are set as shown below:

feed-air flap about 20% (about 225 m³/h),
exhaust-air flap about 25%,
feed-air temperature about 70° C.,
nozzle: as two-component nozzle 1.2 mm in top down, upper nozzle position,
spray pressure 3.5 bar,
spray rate: increasing from 0.02 kg/minute to 0.12 kg/minute,
exhaust-air temperature setting about 40° C.

When the spraying is complete, the resultant material is dried for about a further 10 to 20 minutes in the fluidised bed, with the temperature of the feed air being set so that the product temperature increases to 50° C.

### 2. Main Spraying:

0.50 kg of mannitol and 0.25 kg of sorbitol are dissolved in 2.50 kg of demineralised water with stirring. 1.75 kg of anhydrous calcium hydrogenphosphate are added to the clear solution and suspended. The suspension obtained is stirred for about a further hour in order to destroy any agglomerates formed.

0.5 kg of the pre-spraying is initially introduced in the fluidised-bed granulator (GPCG 5), and the suspension is—as described above with reference to the preparation of the pre-spraying—sprayed on.

This first main spraying can be followed by a plurality of further sprayings, where in each case only a small part of the preceding spraying is initially introduced in the fluidised bed for each spraying, for example 0.5 kg as described above. In this way, the “non-co-sprayed” content in the product is continuously reduced.

The complete drying of the product is checked via the complexometric calcium determination and via the determination of the drying loss over 3 h at 105° C. (as in-process check).

It is of course significantly simpler if co-sprayed material of the desired composition can be taken from preceding sprayings as starting granules and initially introduced in the fluidised bed—the spraying is then carried out as described under point 2 (main spraying).

### Experimental Results:

The results achieved by the various experiments are shown in Tables 1-5 below.

Table 1 shows the tested compositions with different proportions by weight of anhydrous calcium hydrogenphosphate, mannitol and sorbitol.

Table 2 contains the physical data determined for the tested compositions.

Table 3 shows the tabletting data, pressing force, tablet hardness, friability, disintegration time, ejection force of the prepared and tested compositions.

Table 4 shows the corresponding physical data for commercially available anhydrous and directly compressible (DC) calcium hydrogenphosphates and a tabletted mechanical mixture of anhydrous DC calcium hydrogenphosphate, DC mannitol and DC sorbitol.

Table 5 shows the corresponding tabletting data for commercially available anhydrous DC calcium hydrogenphosphates compared with those of the particularly preferred co-spray-granulated combinations consisting of anhydrous calcium hydrogenphosphate, mannitol and sorbitol of Examples C, D and E and with a corresponding mechanical mixture.

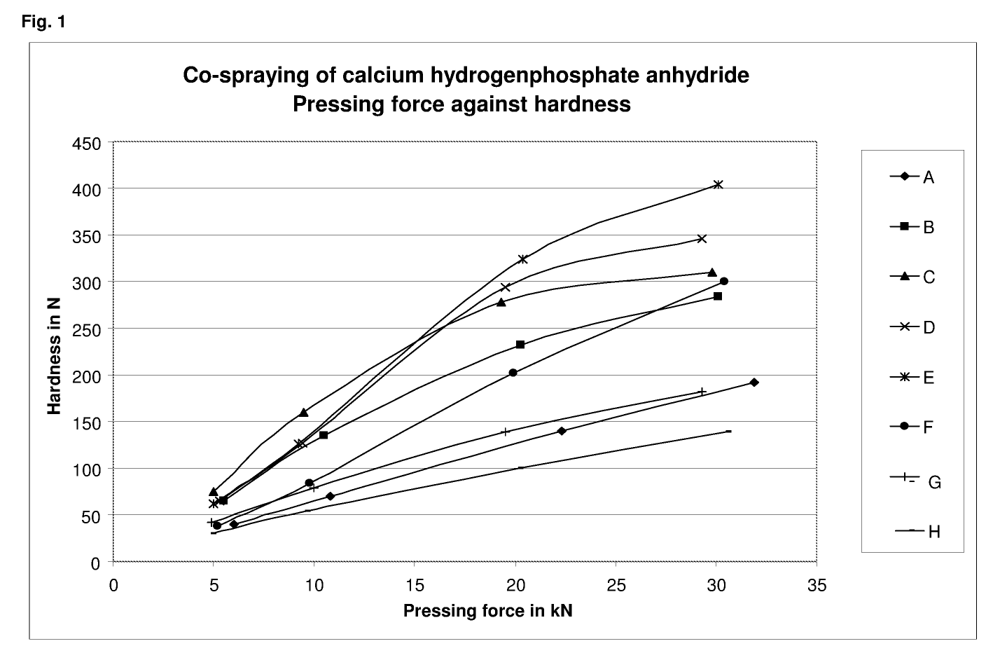

FIG. 1 shows a comparison of the pressing force/hardness profiles for the co-sprayed compositions investigated comprising anhydrous calcium hydrogenphosphate, mannitol and sorbitol. It can be seen from the profiles that on pressing of a co-spray-granulated, anhydrous calcium hydrogenphosphate with 5% by weight of each of mannitol and sorbitol (Example H) to give tablets with increasing pressing pressure, the hardness only varies between about 30 and 140 N. If, however, a co-sprayed composition comprising 70% by weight of anhydrous calcium hydrogenphosphate and 30% by weight of mannitol is tabletted with increasing pressing pressure under the same conditions, products having hardnesses of between about 40 and about 200 N are obtained. For the other compositions investigated, tablets having even higher hardnesses are obtained under the same conditions. The highest hardnesses are achieved, in particular, for compositions in which the calcium hydrogenphosphate:mannitol:sorbitol weight ratio is 50:40:10 or 60:30:10 or 70:20:10.

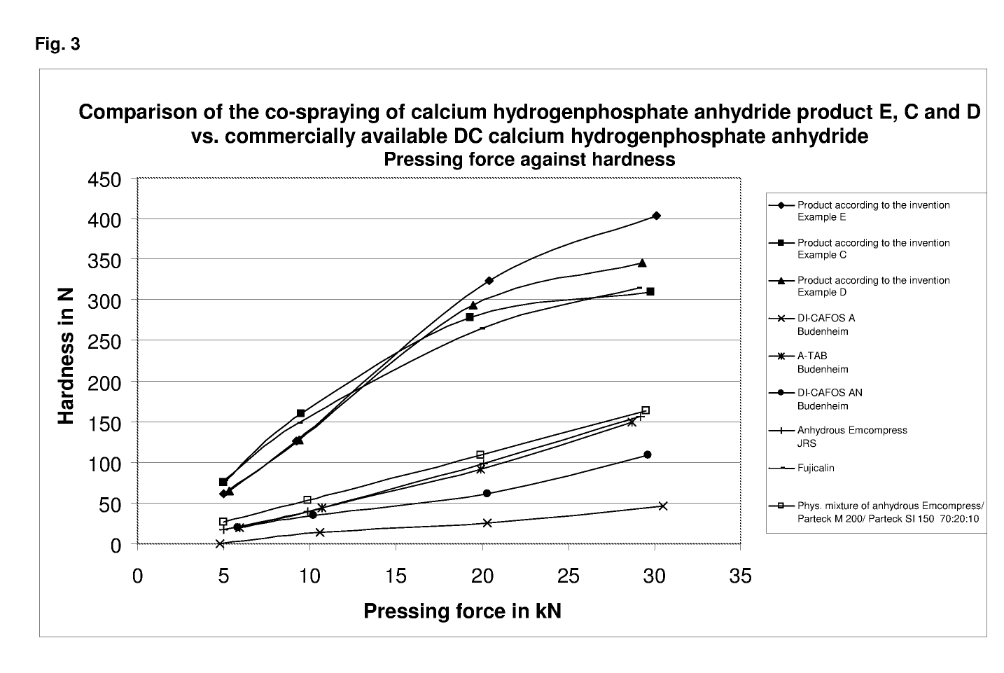

As shown in FIG. 3, a comparison of the compositions according to the invention with the tabletting properties of commercially available directly tablettable calcium hydrogenphosphate anhydride grades shows that, in particular for compositions in which the calcium hydrogenphosphate:mannitol:sorbitol weight ratio is 50:40:10, 60:30:10 or 70:20:10, considerably higher tablet hardnesses are achieved at the same pressing forces, with the exception of Fujicalin, with which comparable hardnesses are achieved than for mixture C, in which calcium hydrogenphosphate, mannitol and sorbitol are co-spray-granulated with one another in the weight ratio 50:40:10. However, it must be noted here that a significantly higher ejection force is necessary on use of Fujicalin for similar tablet hardnesses as for mixture C. By comparison, equally low tablet hardnesses are obtained on pressing of physical mixtures of DC calcium hydrogenphosphate anhydride, DC mannitol and DC sorbitol (70:20:10) as on pressing of commercially available DC calcium hydrogenphosphate anhydrides.

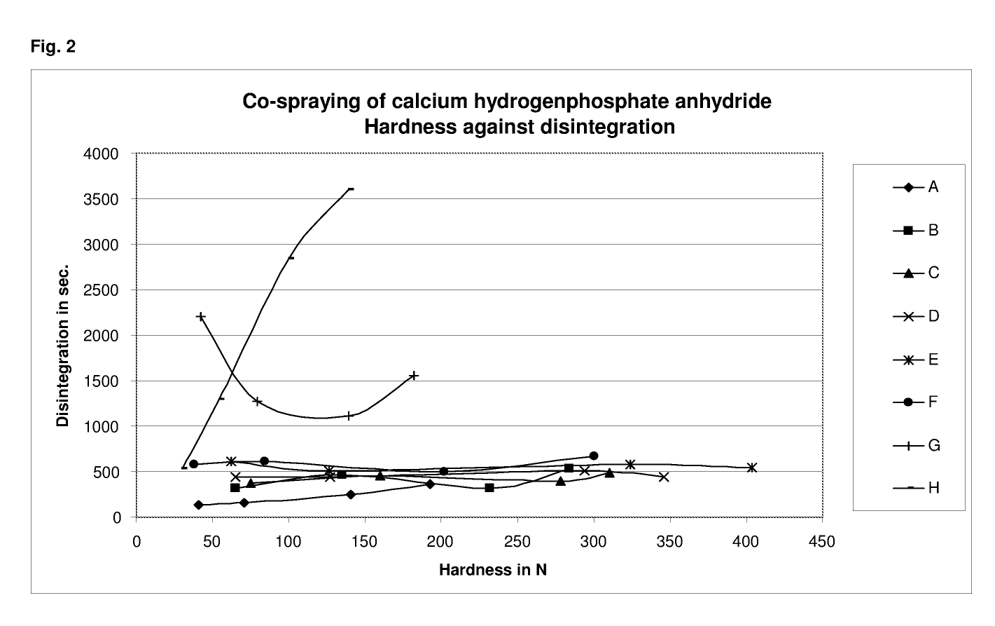

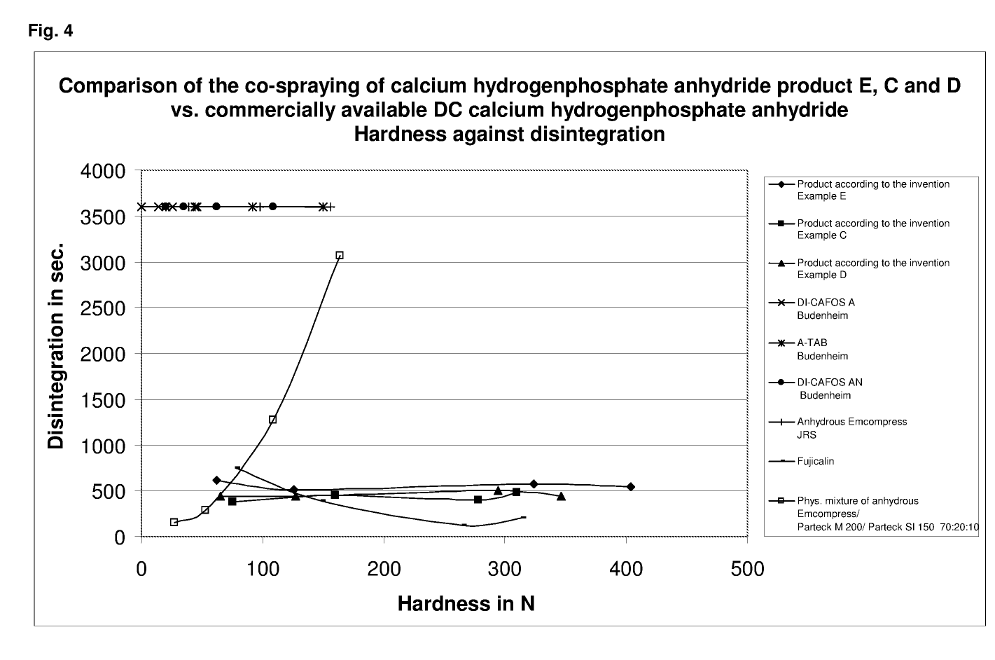

In spite of the increased tablet hardnesses, the corresponding tablets of the compositions according to the invention have very short disintegration times compared with the pure substance, as can be seen very well from the graphical representations in FIG. 2 and FIG. 4. Whereas a tabletted composition consisting of co-spray-granulated calcium hydrogenphosphate anhydride, mannitol and sorbitol in the weight ratio 90:5:5 exhibits an enormously increased disintegration time of up to more than 3600 sec with increasing hardness of between 30 and 139 N, the compositions according to the invention have only disintegration times in the range from about 140 sec to about 670 sec, in spite of increasing hardness, apart from disintegration times of about 1100 to 2200 sec for pressed compositions prepared from a co-spray-granulated composition comprising 85% by weight of calcium hydrogenphosphate anhydride and 10% by weight of mannitol and 5% by weight of sorbitol. As can be seen from FIG. 4, commercially available products exhibit disintegration times of more than 3600 sec compared with the co-spray-granulated compositions according to the invention after pressing to give tablets having a hardness of up to 156 N, with the exception of Fujicalin, which, like the products according to the invention, has fairly short disintegration times even at higher tablet hardnesses, where, however, it must be taken into account that ejection forces up to five times higher are necessary in the case of pressing of Fujicalin to give tablets compared with those on use of the compositions according to the invention.

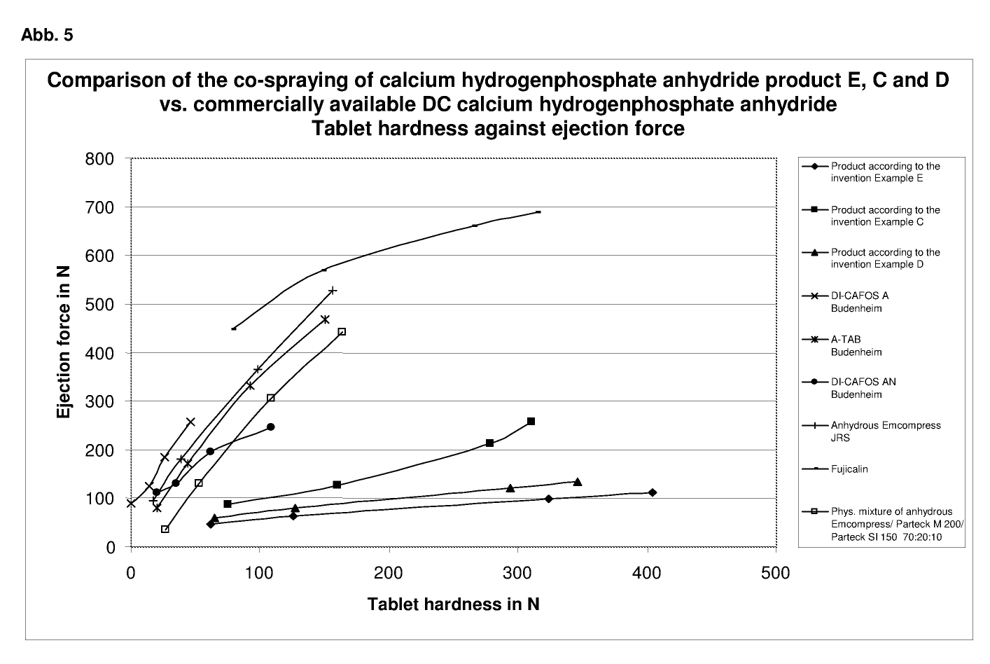

In FIG. 5, the hardnesses of the tablets produced are plotted against the ejection forces. By way of example here, the hardnesses of tablets produced from compositions of Examples C, D and E and the associated ejection forces are compared with those of corresponding commercially available products. This comparison shows quite vividly that tablets made from compositions according to the invention can be ejected from the tabletting moulds with ejection forces which increase relatively little, in spite of increasing hardness. The corresponding comparative data are also shown in Table 5. By contrast, the requisite ejection forces for the commercially available products compared increase very considerably, even with a fairly small increase in the tablet hardness. Accordingly, the stressing of the tabletting machines is significantly less on use of the directly compressible compositions according to the invention compared with the use of commercially available compositions. However, the same also applies to mechanical mixtures of calcium hydrogenphosphate anhydride with the polyols mannitol and sorbitol in the ratio 70:20:10. As already mentioned earlier, commercially available Fujicalin requires particularly high ejection forces after pressing, particularly in this connection.

TABLE 1

| | Calcium hydrogen- | | Sorbitol |
|---|---|---|---|
| | phosphate anhydride | Mannitol | (Parteck SI 400) |
| | Art. No.: 1.2144 | Art. No.: 1.05980 | Art. No.: 1.03140 |
| | Merck KGaA, | Merck KGaA, | Merck KGaA, |
| Product | Germany | Germany | Germany |
| A | 70 | 30 | |
| B | 70 | | 30 |
| C | 50 | 40 | 10 |
| D | 60 | 30 | 10 |
| E | 70 | 20 | 10 |
| F | 80 | 15 | 5 |
| G | 85 | 10 | 5 |
| H | 90 | 5 | 5 |

TABLE 2

| | | A | B | C | D | E | F | G | H |
|---|---|---|---|---|---|---|---|---|---|
| Bulk density [g/ml] | | 0.86 | 0.71 | 0.61 | 0.72 | 0.70 | 0.92 | 0.93 | 0.93 |
| Tapped density | | 1.24 | 0.87 | 0.78 | 0.86 | 0.87 | 1.11 | 1.13 | 1.15 |
| [g/ml] | | | | | | | | | |
| Flow angle [°] | | 41.1 | 32.6 | 32.6 | 29.8 | 32.0 | 32.3 | 32.1 | 32.1 |
| Hausner factor | | 1.44 | 1.23 | 1.28 | 1.19 | 1.24 | 1.21 | 1.22 | 1.24 |
| Compressibility | | 30.65 | 18.39 | 21.80 | 16.28 | 19.54 | 17.12 | 17.70 | 19.13 |
| index [%] | | | | | | | | | |
| Particle-size | | | | | | | | | |
| distribution | | | | | | | | | |
| (in % by weight) | | | | | | | | | |
| <32 | μm | 14.07 | 0 | 0.05 | 0.05 | 0.28 | 0.58 | 2.44 | 1.48 |
| 32-50 | μm | 35.15 | 0.02 | 0.52 | 3.53 | 2.56 | 21.99 | 23.67 | 18.24 |
| 50-100 | μm | 20.51 | 0.76 | 8.83 | 12.53 | 11.13 | 67.81 | 56.33 | 57.10 |
| 100-200 | μm | 24.80 | 3.25 | 32.72 | 23.64 | 59.50 | 7.92 | 12.42 | 15.00 |
| 200-315 | μm | 2.49 | 57.87 | 49.87 | 40.96 | 24.34 | 0.69 | 2.78 | 3.68 |
| 315-500 | μm | 0.98 | 36.61 | 7.69 | 19.02 | 1.90 | 0.44 | 1.70 | 2.97 |
| 500-710 | μm | 0.46 | 1.35 | 0.22 | 0.20 | 0.20 | 0.31 | 0.37 | 1.28 |
| 710-1000 | μm | 0.95 | 0.07 | 0.1 | 0.07 | 0.02 | 0.13 | 0.07 | 0.18 |
| >1000 | μm | 0.59 | 0.07 | 0 | 0 | 0.07 | 0.13 | 0.22 | 0.07 |

| | | | | | | | | |
|---|---|---|---|---|---|---|---|---|
| Calcium content [%] | | | | | | | | |
| theoretical | 20.6 | 20.6 | 14.7 | 17.7 | 20.6 | 23.6 | 25.0 | 26.5 |
| found | 20.7 | 20.7 | 14.9 | 17.3 | 20.4 | 23.3 | 25.0 | 26.3 |
| Drying loss | 0.37 | 0.13 | 0.33 | 0.36 | 0.20 | 0.25 | 0.30 | 0.36 |
| 3 h, 105° C. | | | | | | | | |

TABLE 3

| Co- | Pressing force | | Tablet | | Disintegra- | Ejection |
|---|---|---|---|---|---|---|
| sprayed | [kN] | | hardness | Friability | tion time | force |
| product | nominal actual | | [N] | [%] | [sec] | [N] |
| A | 5 | 6.0 | 40 | 84.842 | 141 | 87 |
| | 10 | 10.8 | 71 | 0.828 | 163 | 147 |
| | 20 | 22.3 | 140 | 0.290 | 254 | 314 |
| | 30 | 31.9 | 193 | 0.212 | 366 | 441 |
| B | 5 | 5.5 | 65 | 0.706 | 315 | 71 |
| | 10 | 10.5 | 135 | 0.275 | 467 | 106 |
| | 20 | 20.3 | 232 | 0.158 | 313 | 172 |
| | 30 | 30.1 | 284 | 0.130 | 538 | 226 |
| C | 5 | 5.0 | 75 | 0.456 | 376 | 87 |
| | 10 | 9.5 | 160 | 0.202 | 449 | 127 |
| | 20 | 19.3 | 278 | 0.139 | 396 | 213 |
| | 30 | 29.8 | 310 | 0.125 | 486 | 258 |
| D | 5 | 5.3 | 65 | 0.202 | 441 | 60 |
| | 10 | 9.4 | 127 | 0.139 | 445 | 80 |
| | 20 | 19.5 | 294 | 0.123 | 507 | 121 |
| | 30 | 29.3 | 346 | 0.117 | 443 | 134 |
| E | 5 | 5.0 | 62 | 0.212 | 617 | 47 |
| | 10 | 9.2 | 126 | 0.151 | 510 | 64 |
| | 20 | 20.4 | 324 | 0.152 | 574 | 99 |
| | 30 | 30.1 | 404 | 0.134 | 540 | 112 |
| F | 5 | 5.2 | 38 | 0.545 | 581 | 38 |
| | 10 | 9.8 | 84 | 0.200 | 609 | 61 |
| | 20 | 19.9 | 202 | 0.119 | 502 | 105 |
| | 30 | 30.4 | 300 | 0.125 | 671 | 145 |
| G | 5 | 4.9 | 42 | 1.364 | 2200 | 87 |
| | 10 | 10.0 | 79 | 0.470 | 1273 | 113 |
| | 20 | 19.5 | 139 | 0.269 | 1113 | 160 |
| | 30 | 29.3 | 182 | 0.191 | 1560 | 204 |
| H | 5 | 4.9 | 30 | 50.941 | 529 | 29 |
| | 10 | 9.6 | 54 | 0.661 | 1300 | 52 |
| | 20 | 20.2 | 100 | 0.318 | 2846 | 106 |
| | 30 | 30.5 | 139 | 0.208 | >3600 | 159 |

TABLE 4

| | | | | | | | Anhydrous |
|---|---|---|---|---|---|---|---|
| | | | | | | | Emcompress/ |
| | | | | | | | Parteck M 200/ |
| | | | | DI- | Anhydrous | | Parteck SI 150 |
| | | DI-CAFOS | | CAFOS | Emcom- | | 70:20:10 mech. |
| | | A | A-TAB | AN | press | Fujicalin | mixture |
| Bulk density (g/ml) | | 1.34 | 0.71 | 0.77 | 0.72 | 0.46 | 0.66 |
| Tapped density | | 1.56 | 0.91 | 0.93 | 0.89 | 0.53 | 0.81 |
| (g/ml) | | | | | | | |
| Flow angle (°) | | 26.2 | 32.3 | 29.1 | 31.8 | 24.7 | 30.6 |
| Hausner factor | | 1.16 | 1.28 | 1.21 | 1.24 | 1.15 | 1.23 |
| Compressibility | | 14.10 | 21.98 | 17.20 | 19.10 | 13.21 | 18.52 |
| index (%) | | | | | | | |
| Particle-size | | | | | | | |
| distribution (in % | | | | | | | |
| by weight) | | | | | | | |
| <32 | μm | 2.78 | 0.11 | 0 | 0.63 | 0 | 0.72 |
| 32-50 | μm | 12.98 | 4.14 | 0.15 | 3.59 | 1.55 | 3.37 |
| 50-100 | μm | 80.03 | 27.37 | 18.28 | 22.38 | 23.40 | 22.09 |
| 100-200 | μm | 3.16 | 45.60 | 59.16 | 48.11 | 73.26 | 46.36 |
| 200-315 | μm | 0.53 | 22.27 | 21.95 | 24.63 | 1.74 | 23.20 |
| 315-500 | μm | 0.32 | 0.45 | 0.38 | 0.52 | 0.05 | 2.94 |
| 500-710 | μm | 0.16 | 0.06 | 0.08 | 0.12 | 0 | 1.03 |
| 710-1000 | μm | 0.04 | 0 | 0 | 0.02 | 0 | 0.12 |
| >1000 | μm | 0 | 0 | 0 | 0 | 0 | 0.17 |

TABLE 5

| | | | | | Disin- | |
|---|---|---|---|---|---|---|
| | Pressing force | | Tablet | | tegration | Ejection |
| | [kN] | | hardness | Friability | time | force |
| Product | nominal actual | | [N] | [%] | [sec.] | [N] |
| Product | 5 | 5 | 62 | 0.212 | 617 | 47 |
| according to | 10 | 9.2 | 126 | 0.151 | 510 | 64 |
| the invention | 20 | 20.4 | 324 | 0.152 | 574 | 99 |
| Ex. E | 30 | 30.1 | 404 | 0.134 | 540 | 112 |
| Product | 5 | 5.0 | 75 | 0.456 | 376 | 87 |
| according to | 10 | 9.5 | 160 | 0.202 | 449 | 127 |
| the invention | 20 | 19.3 | 278 | 0.139 | 396 | 213 |
| Ex. C | 30 | 29.8 | 310 | 0.125 | 486 | 258 |
| Product | 5 | 5.3 | 65 | 0.202 | 441 | 60 |
| according to | 10 | 9.4 | 127 | 0.139 | 445 | 80 |
| the invention | 20 | 19.5 | 294 | 0.123 | 507 | 121 |
| Ex. D | 30 | 29.3 | 346 | 0.117 | 443 | 134 |
| DI-CAFOS A | 5 | 4.8 | 0 | 100 | >3600 | 90 |
| Budenheim | 10 | 10.6 | 14 | 100 | >3600 | 125 |
| | 20 | 20.3 | 26 | 100 | >3600 | 185 |
| | 30 | 30.5 | 46 | 65.26 | >3600 | 258 |
| A-TAB | 5 | 5.9 | 20 | 1.780 | >3600 | 80 |
| Budenheim | 10 | 10.7 | 44 | 0.502 | >3600 | 171 |
| | 20 | 19.9 | 92 | 0.275 | >3600 | 332 |
| | 30 | 28.7 | 150 | 0.176 | >3600 | 468 |
| DI-CAFOS AN | 5 | 5.8 | 20 | 100 | >3600 | 111 |
| Budenheim | 10 | 10.2 | 35 | 27.962 | >3600 | 131 |
| | 20 | 20.3 | 62 | 0.968 | >3600 | 196 |
| | 30 | 29.6 | 109 | 0.226 | >3600 | 246 |
| Anhydrous | 5 | 5.0 | 17.4 | 36.899 | >3600 | 95 |
| Emcompress | 10 | 9.9 | 39.2 | 0.571 | >3600 | 181 |
| JRS | 20 | 20.1 | 98.2 | 0.257 | >3600 | 365 |
| | 30 | 29.2 | 156.3 | 0.162 | >3600 | 528 |
| Fujicalin | 5 | 5.0 | 78 | 0.018 | 752 | 448 |
| | 10 | 9.3 | 148 | 0.045 | 394 | 568 |
| | 20 | 19.9 | 265 | 0.021 | 120 | 660 |
| | 30 | 29.0 | 314 | 0.106 | 205 | 688 |
| Phys. mixture | 5 | 5.0 | 27 | 1.306 | 151 | 35 |
| of anhydrous | 10 | 9.9 | 53 | 0.365 | 289 | 130 |
| Emcompress/ | 20 | 19.9 | 109 | 0.200 | 1274 | 306 |
| Parteck M200/ | 30 | 29.5 | 164 | 0.168 | 3065 | 442 |
| Parteck SI150 | | | | | | |
| 70:20:10 | | | | | | |

1.-20. (canceled)

21. A directly compressible tabletting composition, comprising 50-85% by weight of anhydrous calcium hydrogenphosphate, 10-40% by weight of mannitol and 5-20% by weight of sorbitol, wherein said composition has a flow angle in the range of 29 to 33.4° and a bulk density in the range of 0.56 to 0.77 g/ml with a tapped density in the range of 0.73 to 0.92 g/ml.

22. The directly compressible tabletting composition according to claim 21, further comprising one or more homogeneously distributed, water-insoluble and/or water-soluble additives.

23. The directly compressible tabletting composition according to claim 21, one or more one or more homogeneously distributed, water-insoluble and/or water-soluble additives selected from: pharmaceutical active compounds, plant extracts, sweeteners, dyes, citric acid, vitamins and trace elements.

24. The directly compressible tabletting composition according to claim 23, which comprises one or more pharmaceutical active compounds which are analgesics.

25. The directly compressible tabletting composition according to claim 23, which comprises one or more sweeteners selected from: acesulfame K, Aspartame®, saccharin, cyclamate, sucralose and neohesperidin DC.

26. The directly compressible tabletting composition according to claim 21, which comprises 60 to 80% by weight of anhydrous calcium hydrogenphosphate, 15 to 25% by weight of mannitol and 7 to 13% by weight of sorbitol.

27. The directly compressible tabletting composition according to claim 21, which comprises 65 to 75% by weight of anhydrous calcium hydrogenphosphate, 17 to 23% by weight of mannitol and 8 to 12% by weight of sorbitol.

28. The directly compressible tabletting composition according to claim 21, wherein the anhydrous calcium hydrogenphosphate, mannitol and sorbitol are co-spray granulated.

29. The directly compressible tabletting composition according to claim 21, wherein the composition has a particle-size distribution of max. 3% by weight of undersized particles having a particle size of <32 μm, max. 5% by weight of oversized particles having a particle size of >500 μm, and 50 to 90% by weight of a particle fraction having particle sizes in the range from 100 to 315 μm.

30. The directly compressible tabletting composition according to claim 21, wherein the composition has a calcium content of 14 to 21% by weight, based on the total amount, and a drying loss of less than 2% by weight.

31. The directly compressible tabletting composition according to claim 21, wherein the composition has a calcium content of 14 to 21% by weight, based on the total amount, and a drying loss of less than 1% by weight.

32. The directly compressible tabletting composition according to claim 21, wherein the composition is in the form of tablets formed by compression with a pressing force of 20 kN, and the tablets have a hardness of >270 N, together with an ejection force of <215 N, a friability of <0.16%, and a disintegration time of <580 seconds.

33. The directly compressible tabletting composition according to claim 21, wherein the composition is in the form of tablets formed by compression with a pressing force of 20 kN, and the tablets have a hardness of >300 N, together with an ejection force of <100 N, a friability of <0.16% and a disintegration time of <580 seconds.

34. The directly compressible tabletting composition according to claim 21, wherein the composition is in the form of tablets formed by compression with a pressing force of 30 kN, and the tablets have a hardness of >350 N, together with an ejection force of <115 N, a friability of <0.14% and a disintegration time of <550 seconds.

35. The directly compressible tabletting composition according to claim 21, wherein the composition is in the form of tablets formed by compression.

36. A process for preparing the directly compressible tabletting composition according to claim 21, which comprises dissolving or suspending a solution or suspension comprising 50 to 85% by weight of anhydrous calcium hydrogenphosphate, 10 to 40% by weight of mannitol and 5 to 20% by weight of sorbitol in water, where 4 parts of solid are dissolved or suspended in 4 parts of water, and subjecting the solution or suspension to a co-spray-granulation process, either batchwise or continuously, in a fluidised-bed granulator.

37. A process for preparing the directly compressible tabletting composition according to claim 26, which comprises dissolving or suspending a solution or suspension comprising 60 to 80% by weight of anhydrous calcium hydrogenphosphate, 15 to 25% by weight of mannitol and 7 to 13% by weight of sorbitol in water, where 4 parts of solid are dissolved or suspended in 4 parts of water, and subjecting the solution or suspension to a co-spray-granulation process, either batchwise or continuously, in a fluidised-bed granulator.
